(19) United States
(12) Patent Application Publication
(10) Pub. No.: US 2025/0059449 A1
(43) Pub. Date: Feb. 20, 2025

(54) Ethanol Conversion Using FCC System

(71) Applicant: ExxonMobil Technology and Engineering Company, Baytown, TX (US)

(72) Inventors: Jihad M. Dakka, Whitehouse Station, NJ (US); Arun K. Sharma, Hellertown, PA (US); Xiaochun Xu, Basking Ridge, NJ (US); Richard C. Senior, Spring, TX (US); Jason M. Golias, Clinton, NJ (US)

(21) Appl. No.: 18/744,973

(22) Filed: Jun. 17, 2024

Related U.S. Application Data
(60) Provisional application No. 63/520,138, filed on Aug. 17, 2023.

(51) Int. Cl. C10G 11/05; C07C 1/24; C10G 11/18

(52) U.S. Cl. CPC C10G 11/05; C07C 1/24; C10G 11/18; C07C 2529/06; C10G 2400/20

(57) ABSTRACT

The present disclosure provides methods and systems for converting ethanol and cracking hydrocarbons into ethylene and at least cracked products. A method to convert ethanol and cracking hydrocarbons includes: introducing a hydrocarbon feed into a riser of a fluid catalytic cracking reactor such that hydrocarbons in the hydrocarbon feed reacts in the presence of one or more fluid catalytic cracking catalysts to produce at least cracked products; and introducing an ethanol feed into an upper one third of a stripper of the fluid catalytic cracking reactor such that the ethanol reacts in the presence of spent catalyst flowing downwardly from a separator section of the fluid catalytic cracking reactor to produce at least ethylene.

### CROSS-REFERENCE TO RELATED APPLICATIONS

This application claims priority to and the benefit of U.S. Provisional Application No. 63/520,138 having a filing date of Aug. 17, 2023, the disclosure of which is incorporated herein by reference in its entirety.

### FIELD

This application relates to processing of an ethanol feed in a fluid catalytic cracking system and, more particularly, one or more embodiments relate to the co-processing in a fluid catalytic cracking system of a hydrocarbon feed and an ethanol feed.

### BACKGROUND

Light olefins such as ethylene, propylene, butenes and any mixtures thereof serve as feeds for the production of numerous important chemicals and polymers. Light olefins traditionally are produced by cracking petroleum feeds. Because of the limited supply and escalating cost of petroleum feeds, the cost of producing olefins from petroleum sources has increased steadily. Efforts to develop and improve olefin production technologies, particularly light olefins production technologies, based on alternative feed stocks have increased.

An important type of alternative feedstock for the production of light olefins are oxygenates, such as alcohols, particularly methanol and ethanol. Alcohols may be produced by fermentation, or from synthesis gas derived from natural gas, petroleum liquids, carbonaceous materials including coal, recycled plastics, municipal wastes, agricultural products, or most organic materials. Because of the wide variety of raw material sources, alcohol, alcohol derivatives, and other oxygenates have promise as an economical, non-petroleum feedstock source for olefin production.

Cracking of hydrocarbons is a process that is widely used to break down larger and higher boiling hydrocarbons into smaller and lower boiling hydrocarbons that can be more valuable. Fluid catalytic cracking (FCC) is one technique for hydrocarbon cracking that uses catalyst and heat to break down larger and higher boiling hydrocarbons into smaller and lower boiling hydrocarbons, such as naphtha, gasoline, distillate, and other petroleum products. Typically, the feedstock for FCC is a heavy gas oil with the heavy gas oil heated then placed into contact with a catalyst that breaks apart the larger hydrocarbons into smaller molecules. Conventional FCC, however, can be sensitive to alternative feedstocks. For example, co-feeding alternative feedstocks into an FCC system can result in undesirable conversion rates. It would be desirable, however, to have improved processes and systems for processing alternative feedstocks in FCC systems.

### SUMMARY

Disclosed herein is an example of a method to convert ethanol and cracking hydrocarbons comprising: introducing a hydrocarbon feed into a riser of a fluid catalytic cracking reactor such that hydrocarbons in the hydrocarbon feed reacts in the presence of one or more fluid catalytic cracking catalysts to produce at least cracked products; and introducing an ethanol feed into an upper one third of a stripper of the fluid catalytic cracking reactor such that the ethanol reacts in the presence of spent catalyst flowing downwardly from a separator section of the fluid catalytic cracking reactor to produce at least ethylene.

Further disclosed herein is an example of a system for ethanol conversion and cracking hydrocarbons, comprising: a first source of a hydrocarbon feed comprising hydrocarbons; a second source of an ethanol feed comprising ethanol; and a fluid catalytic cracking system comprising a fluid catalytic cracking reactor and a catalyst regenerator, wherein the fluid catalytic cracking reactor is fluidically coupled to the catalyst regenerator such that the fluid catalytic cracking reactor receives regenerated catalyst from the catalyst regenerator, wherein the fluid catalytic cracking reactor comprises a riser fluidically coupled to the first source of the hydrocarbon feed; and wherein the fluid catalytic cracking reactor comprises a reactor vessel that comprises a separator section and a stripper, the stripper being fluidically coupled to the second source of the ethanol feed.

These and other features and attributes of the disclosed methods and systems of the present disclosure and their advantageous applications and/or uses will be apparent from the detailed description which follows.

### BRIEF DESCRIPTION OF THE DRAWINGS

To assist those of ordinary skill in the relevant art in making and using the subject matter hereof, reference is made to the appended drawings, wherein:

FIG. 1 is an illustrative depiction of a fluid catalytic cracking (FCC) system for processing a hydrocarbon feed and an ethanol fee in accordance with certain embodiments of the present disclosure.

FIG. 2 is an illustrative depiction of a pilot plant system used to co-process various reactor feeds in accordance with certain embodiments of the present disclosure.

FIG. 3 is a chart of ethylene and ethane yield for various feeds to the pilot plant system of FIG. 2 in accordance with certain embodiments of the present disclosure.

FIG. 4 is a chart of coke yield as a function of feed in accordance with certain embodiments of the present disclosure.

FIG. 5 is a chart of carbon monoxide and carbon dioxide yield as a function in accordance with certain embodiments of the present disclosure.

FIG. 6 is a chart of ethylene yield as a function of catalyst in accordance with certain embodiments of the present disclosure.

FIG. 7 is a chart of ethane yield as a function of catalyst in accordance with certain embodiments of the present disclosure.

FIG. 8 is a chart of carbon monoxide plus carbon dioxide yield as a function of catalyst in accordance with certain embodiments of the present disclosure.

FIG. 9 is a chart of the propylene (C3) yield as a function of catalyst in accordance with certain embodiments of the present disclosure.

FIG. 10 is a chart of the other hydrocarbon product (C₄₊) yield as a function of catalyst in accordance with certain embodiments of the present disclosure.

FIG. 11 is a chart of liquid analysis indicating that mainly diethyl ether was formed in addition to water in accordance with certain embodiment of the present disclosure.

### DETAILED DESCRIPTION

This application relates to methods and systems for co-processing a hydrocarbon feed and an ethanol feed in a fluid catalytic cracking (FCC) system. Advantageously, ethanol is a non-petroleum source and can be converted into ethylene in an FCC system, for example, using FCC reactor, FCC catalyst and/or typical FCC conditions. Further, the use of existing facilities and processes improves the economics of ethylene production from ethanol. The ethanol dehydration reaction is carried out in vapor phase using fixed bed or fluidized bed reactor in accordance with one or more embodiments. For fixed bed reactors, the operation can be either isothermal or adiabatic. In the isothermal design, weak acid catalysts such as alumina or silica-alumina can be used at temperatures of 300° C. to 350° C., for example, which gives an ethanol conversion in between 94% and 97%. In some embodiments, the typical catalyst lifecycle is between 3 weeks to 4 months followed by regeneration for up to 3 days. The fluidized-bed process offers excellent temperature control and excellent selectivity with up to a 99.6% mole of ethanol conversion, in accordance with example embodiments.

In accordance with present embodiments, ethanol dehydration is performed in an FCC reactor. However, feeding ethanol into any location in the FCC reactor can be problematic. For instance, ethanol and vacuum gas oil (VGO) can be fed into the riser in the FCC reactor. However, the presence of VGO in the riser alongside ethanol results in significant ethylene to ethane conversion through hydrogen transfer as shown in the following reaction schematic:

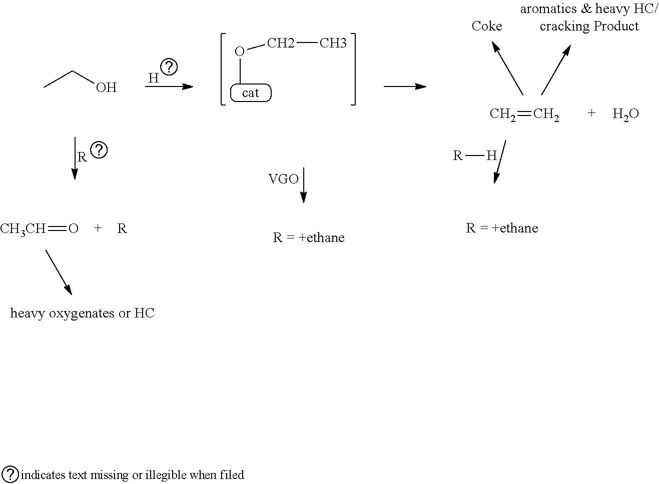

This is undesirable as the hydrogen transfer illustrated in the reaction schematic above reduces the selectivity toward ethylene. In contrast, example embodiments disclosed herein relate to methods and systems for ethanol dehydration using FCC systems at high conversion/selectivity, while still using a conventional hydrocarbon feed, such as VGO. Accordingly, example embodiments feed the ethanol to the stripper, upstream from the riser, to avoid a long contact time between the ethanol, the catalyst and the hydrocarbon, thus avoiding the conversion of ethylene to ethane through hydrogen transfer discussed above. Further, ethanol injected in the stripper is in contact with a catalyst at least partly free of hydrocarbons after flowing through a separator section (e.g., cyclone separator) within the reactor. In the stripper, the spent catalyst flows downward through a stripper to remove any hydrocarbon vapors before the spent catalyst returns to the catalyst regenerator.

### FCC System Feeds

Example embodiments include processing of a hydrocarbon feed including hydrocarbons in an FCC system. The hydrocarbon feed includes any of a variety of suitable hydrocarbons that can be processed in an FCC system. Examples of suitable hydrocarbon feeds include whole and reduced petroleum crudes, atmospheric and vacuum residue, propane deasphalted residue, e.g., brightstock, cycle oils, fluid catalytic tower bottoms, gas oils, including atmospheric and vacuum gas oils and coker gas oils, light to heavy distillates including raw virgin distillates, hydrocrackates, hydrotreated oils, dewaxed oils, slack waxes, Fischer-Tropsch waxes, raffinates, and mixtures of these materials. In some embodiments, the hydrocarbon feed includes vacuum gas oils boiling up to 1100° F. (593° C.), including vacuum gas oils boiling in the range of 660° F. to 935° F. (350° C. to 500° C.), as determined in accordance with ASTM D2887, titled “Standard Test Method for Boiling Range Distribution of Petroleum Fractions by Gas Chromatograph.” The hydrocarbon feed is fed to the FCC system at any suitable concentration, including in an amount of 50% or more by weight of a combined feed of the hydrocarbon feed and ethanol feed. In some embodiments, the hydrocarbon feed is fed to the FCC system at any suitable concentration, including in an amount of 50 wt. % or more of a combined feed. For example, the hydrocarbon feed is fed to the FCC system at concentrations of 50 wt. % to 95 wt. %, 60 wt. % to 95 wt. %, 70 wt. % to 95 wt. %, 75 wt. % to 95 wt. %, 80 wt. % to 95 wt. %, 50 wt. % to 90 wt. %, 50 wt. % to 80 wt. %, 50 wt. % to 70 wt. %, 50 wt. % to 60 wt. %, 60 wt. % to 90 wt. %, 70 wt. % to 90 wt. %, 80 wt. % to 90 wt. %, 70 wt. % to 85 wt. %, or 75 wt. % to 85 wt. % of the combined feed, or any ranges therebetween.

As previously described, the hydrocarbon feed including hydrocarbons is fed to the riser of the FCC system while the ethanol feed is fed into the stripper, above the riser, of the FCC system. The ethanol used in the example embodiments herein includes ethanol from any suitable source. In some embodiments, the ethanol is produced from plant material (e.g., fractionated plant material) such as corn. For example, ethanol is produced by fractionating the plant material; grinding the plant material (e.g., fractionated plant material) to produce ground plant material including starch; saccharifying the starch, without cooking; fermenting the incubated starch; and recovering the ethanol from the fermentation. In some embodiments, temperature is varied during fermentation. In alternative embodiments, ethanol is produced from synthesis gas derived from natural gas, petroleum liquids, carbonaceous materials, including coal, recycled plastics, municipal wastes, or other suitable organic material. For example, the ethanol is produced from a homologation reaction of methanol with carbon monoxide and optionally hydrogen. Because of the wide variety of sources, alcohol, alcohol derivatives, and other oxygenates have promise as economical, non-petroleum sources for light olefin production.

The ethanol is fed to the FCC system at any suitable concentration, including in an amount of 50% or less by weight of a combined feed of the hydrocarbon feed and ethanol feed. In some embodiments, the ethanol feed is fed to the FCC system at any suitable concentration, including in an amount of 50 wt. % or more of a combined feed. For example, the hydrocarbon feed is fed to the FCC system at concentrations of 5 wt. % to 50 wt. %, 5 wt. % to 40 wt. %, 5 wt. % to 30 wt. %, 5 wt. % to 25 wt. %, 5 wt. % to 20 wt. %, 10 wt. % to 50 wt. %, 20 wt. % to 50 wt. %, 30 wt. % to 50 wt. %, 40 wt. % to 50 wt. %, 10 wt. % to 40 wt. %, 10 wt. % 30 wt. %, 10 wt. % to 20 wt. %, 15 wt. % to 30 wt. %, or 15 wt. % to 25 wt. % of the combined feed, or any ranges therebetween.

### Fluid Catalytic Cracking System

In one or more embodiments, the hydrocarbon feed including hydrocarbons is co-processed in an FCC system with an ethanol feed. FCC systems are commonly used in refineries as a method for converting feedstock to produce lower boiling fractions suitable for use as fuels. The process has become the pre-eminent source for motor gasoline in the USA and serves the petrochemical industry with light olefins as petrochemical feedstock. Normal FCC operation cracks large molecules to a wide boiling mixture including olefins (alkenes).

The hydrocarbon feed is introduced into the FCC system and cracked into shorter molecules to produce cracked products. Examples of suitable cracked products include gasoline, diesel, distillate, olefins (e.g., ethylene, propylene, butylene, etc.), and other petroleum products. Typically, the FCC system includes a reactor for contacting the feeds with a fluidized FCC catalyst and a catalyst regenerator for regenerating the spent FCC catalyst for re-use. Conventional fluidized catalytic cracking units are suitable, but the invention is not limited thereto. The hydrocarbon feed can be cracked in the presence of one or more fluidized catalysts to produce a catalytically cracked effluent. A typical FCC reactor is a vessel in which the cracked product vapors are: (a) separated from the spent catalyst by flowing through a set of two-stage cyclones within the reactor and (b) the spent catalyst flows downward through a steam stripping section to remove any hydrocarbon vapors before the spent catalyst returns to the catalyst regenerator. The ethanol feed can be dehydrated into ethylene by feeding the stripper of the FCC reactor. For instance, the vacuum gas oil is fed to the riser, before the cracking zone, while ethanol is fed to the stripper, at the end of the cracking zone of the FCC unit.

The catalytically cracked effluent can be introduced to one or more separators and one, two, or more products can be separated therefrom. In some embodiments, one, two, or more of an FCC C₄₋ product (a light hydrocarbon product), an FCC naphtha, an FCC cycle oil, and a bottoms product are recovered from the one or more separators. The FCC C₄₋ product typically includes C₁, C₂, C₃, and C₄ hydrocarbon, and in addition generally one or more of molecular hydrogen, ammonia, carbon dioxide, arsine, mercury, hydrogen sulfide, carbonyl sulfide, mercaptans, and carbon disulfide, oxygenates or water. The C2 hydrocarbon in the FCC C₄₋ product includes ethylene produce from processing of the ethanol. The FCC system can include additional equipment that is typically used in such a process, e.g., a separator such as a cyclone separator for separating the fluidized catalyst from the catalytically cracked effluent. It should also be understood that the FCC system can also include additional separators such as a catalyst fines separator configured to remove entrained catalyst particles from the bottoms product or other product(s) separated therefrom.

In at least one embodiment, the FCC system includes a riser reactor. In accordance with example, embodiments, the hydrocarbon feed is introduced into the riser reactor. In some embodiments, the riser reactor is an “internal” riser reactor or an “external” riser reactor, such as a vertical tube-shaped reactor, for example, which has a vertical upstream end located outside a reaction vessel and a vertical downstream end located inside the reaction vessel. Examples of suitable reaction vessels include a reaction vessel suitable for catalytic cracking reactions and/or a reaction vessel that includes one or more cyclone separators and/or swirl tubes to separate spent catalyst from cracked product. Examples of suitable reaction vessels also include a stripper in which hydrocarbon vapors are removed from spent catalyst flowing downward from the cyclone separators.

Any number of reactors can be operated in series and/or in parallel. Any two or more types of reactors can be used in combination with one another. If two or more reactors are used, the reactors can be operated at the same conditions and/or different conditions and can receive the same hydrocarbon-containing feed or different hydrocarbon-containing feeds. If two or more reactors are used, the reactors can be arranged in series, in parallel, or a combination thereof with respect to one another. In some embodiments, suitable reactors can be or can include, but are not limited to, high gas velocity riser reactors, high gas velocity downer reactors, vortex reactors, reactors having a relatively dense fluidized catalyst bed at a first or bottom end and a relatively less dense fluidized catalyst within a riser located at a second or top end, multiple riser reactors and/or downer reactors operated in parallel and/or series operating at the same or different conditions with respect to one another, or combinations thereof.

In one or more embodiments, the reactor is designed to have two or more feed injection nozzles. In some embodiments, the hydrocarbon feed including the hydrocarbons is introduced into the riser through separate injection nozzles from the ethanol. In one or more embodiments, the hydrocarbon feed is introduced into the riser through separate injection nozzles from the ethanol feed, which is introduced into the stripper, upstream from the riser. The FCC catalyst can be any suitable catalyst for use in a cracking process. In at least one embodiment, the FCC catalyst includes any suitable zeolitic component for the FCC. Also, example embodiments of FCC catalyst further includes an amorphous binder compound and/or a filler. Examples of the amorphous binder component include quartz, zirconia, silica, alumina, magnesium oxide, calcium carbonate, and/or titania, and/or a mixture thereof of at least two or more of these components. Examples of suitable fillers include clays (such as hydrated aluminum silicate, also called “kaolin”) and/or silica. For purpose of the present disclosure, the zeolitic component can be a large, a medium, and/or a mixture thereof of large and medium pore zeolite which include a porous, crystalline aluminosilicate structure, for example.

In some embodiments, the FCC catalyst can be pneumatically moved through the reactor via a carrier fluid or transport fluid. The transport fluid can be or can include, but is not limited to, a diluent, one or more of the feeds in gaseous form, or a mixture thereof. Examples of suitable transport fluids include molecular nitrogen, volatile hydrocarbons such as methane, ethane, and/or propane, argon, carbon monoxide, carbon dioxide, steam, and the like. The amount of transport fluid can be sufficient to maintain the catalyst in a fluidized state and to transport the catalyst particles from one location, e.g., the combustion zone or the regeneration zone, to a second location, e.g., the conversion zone. In some embodiments, a weight ratio of the catalyst to the transport fluid can be in a range from 1, 5, 10, 15, or 20 to 50, 60, 80, 90, or 100.

The feeds and FCC catalyst can be contacted in the reactor at a reactor operating temperature in a range from 300° C. to 900° C., including from 300° C. to 800° C., from 300° C. to 700° C., 300° C. to 600° C., 300° C. to 500° C., 300° C. to 400° C., 400° C. to 900° C., 400° C. to 800° C., 400° C. to 700° C., 400° C. to 600° C., 500° C. to 900° C., 500° C. to 800° C., or 500° C. to 700° C., or any range therebetween. The feeds can be introduced into the reactor and contacted with the FCC catalyst therein for a time period in a range from 0.1 seconds to 3 minutes, including from 1 second to 3 minutes, from 1 second to 2 minutes, from 1 second to 1 minute, from 1 second to 30 seconds, from 30 seconds to 3 minutes, from 30 seconds to 2 minutes, or from 30 seconds to 1 minute, or any range therebetween.

The average residence time of the FCC catalyst within the conversion zone can be ≤7 minutes, ≤6 minutes, ≤5 minutes, ≤4 minutes ≤3 minutes, ≤2 minutes, ≤1.5 minutes, ≤1 minute, ≤45 seconds, ≤30 seconds, ≤20 seconds, ≤15 seconds, ≤10 seconds, ≤7 seconds, ≤5 seconds, ≤3 seconds, ≤2 seconds, or ≤1 second. In some embodiments, the average residence time of the FCC catalyst within the conversion zone can be greater than an average residence time of the feeds.

The feeds and FCC catalyst can be contacted under a hydrocarbon partial pressure of at least 20 kPa-absolute, where the hydrocarbon partial pressure is the total partial pressure of any C₂-C₁₆ alkanes and any C₈-C₁₆ alkyl aromatic hydrocarbons in the feeds. In some embodiments, the hydrocarbon partial pressure during contact of the feeds and the FCC catalyst is in a range from 20 kPa-absolute to 1,000 kPa-absolute, including from 20 kPa-absolute to 750 kPa-absolute, from 20 kPa-absolute to 500 kPa-absolute, from 20 kPa-absolute to 200 kPa-absolute, from 20 kPa-absolute to 100 kPa-absolute, from 50 kPa-absolute to 1,000 kPa-absolute, from 100 kPa-absolute to 1,000 kPa-absolute, from 200 kPa-absolute to 1,000 kPa-absolute, from 500 kPa-absolute to 1,000 kPa-absolute, or from 750 kPa-absolute to 1,000 kPa-absolute, or any range therebetween, where the hydrocarbon partial pressure is the total partial pressure of any C₂-C₁₆ alkanes and any C₈-C₁₆ alkyl aromatic hydrocarbons in the total feed of the hydrocarbon feed and ethanol feed.

In at least one embodiment, cracked products and ethylene produced in the reactor are separated from spent (deactivated) FCC catalyst. Example embodiments include a catalyst regenerator for regenerating the spent FCC catalyst for re-use. The regenerated FCC catalyst is then recycled to the reactor in accordance with present embodiments. In at least one embodiment, a side stream of make-up FCC catalyst is added to the recycle stream to make-up for loss of FCC catalyst in the reaction zone and regenerator.

Separation of the spent FCC catalyst from the cracked products uses any suitable technique. In some embodiments, the separation includes separating one or more of the cracked products of the hydrocarbon source and ethylene produced from the ethanol source from the spent FCC catalyst in a separation section of the reactor, for example, using one or more cyclone separators and/or one or more swirl tubes. Furthermore, example embodiments include a stripper to recover the cracked products absorbed on the spent FCC catalyst before catalyst regeneration. In some embodiments, the separation section and stripper are in the same reaction vessel. As previously mentioned, the ethanol is fed into the stripper in accordance with one or more embodiments. In some embodiments, a stripping medium (e.g., steam) is fed to the stripper, for example, to counter currently contact spend catalyst downwardly flowing in the stripper. In some embodiments, the recovered products are recycled and added to a stream including one or more cracked products obtained from the catalytic cracking process.

In one or more embodiments, the ethanol is injected into the stripper at any suitable location. Feeding the ethanol too low into the stripper can result in ethylene carryover to the regenerator while feeding the ethanol too high into the stripper can result in complete ethanol conversion. In at least one embodiment, ethanol is injected in the upper third of the stripper, wherein each third is equal in height. In at least one embodiment, ethanol is injected in the second third of the stripper. In at least one embodiment, ethanol is injected in the bottom third of the stripper. In at least one embodiment, ethanol is injected in the upper half of the stripper. In at least one embodiment, ethanol is injected in the lower half of the stripper. In at least one embodiment, ethanol is fed in multiple locations at the same time to create a uniform distribution to minimize downflow of ethylene/unconverted ethanol.

In at least one embodiment, catalyst regeneration includes contacting the spent FCC catalyst with an oxygen-containing gas in the catalyst regenerator, in order to produce a regenerated FCC catalyst, heat, and carbon dioxide. The catalyst activity can be restored during the regeneration where the coke that can be deposited on the FCC catalyst, as a result of the reactions in the reactor, is burned off. Examples of suitable oxygen-containing gases include any suitable oxygen containing gas, such as air or oxygen-enriched air (OEA).

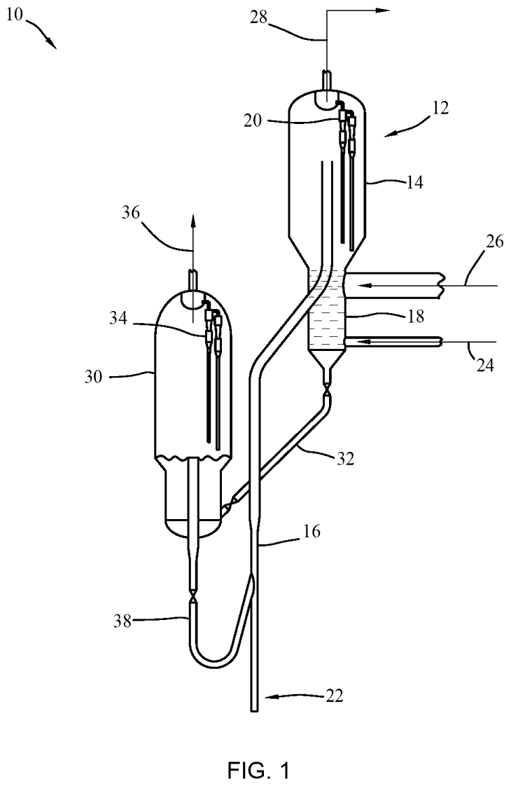

FIG. 1 illustrates an FCC system 10 in accordance with one or more embodiments. The FCC system 10 includes a reactor 12 in the form of a riser reactor that includes a reaction vessel 14 and a riser 16 coupled to the reaction vessel 14. The reaction vessel 12 contains an FCC catalyst (not shown), a stripper 18 and a separator section 20 (e.g., a two-stage cyclone separator), wherein the separator section 20 separates the heavy catalyst from the hydrocarbon products. As illustrated, a hydrocarbon feed 22 is fed to the riser 16 of the reactor 12 where it is vaporized and cracked into smaller molecules by contact and mixing with the FCC catalyst, for example. The reaction in the reactor 12 also produces a carbonaceous material (e.g., coke) that deposits on the FCC catalyst causing it to lose effectiveness. A steam feed 24 is fed to the stripper 18 to remove some of the coke remaining on the spent catalyst after coming down from the separator section 20 and re-activate at least part of it. An ethanol feed 26 is also fed to the stripper 18 where it is dehydrated into ethylene by contact and mixing with the partly re-activated FCC catalyst. As illustrated, the ethanol feed 26 is fed to the stripper 18 above the steam feed 24. By feeding an ethanal fed 26 to the stripper 18, the contact time between ethanol and the partly re-activated FCC catalyst is minimized and the partly re-activated FCC catalyst is not as efficient as a new catalyst. Therefore, the combination of these two factors along with the reaction temperature prevents ethylene from converting to ethane through hydrogen transfer in contrast with the reaction occurring when ethanol is fed through the riser as discussed above.

The location of feed nozzle of the ethanol feed 26 into the stripper 18 can be optimized, for example, to maintain a high yield of conversion from ethanol to ethylene. If the nozzle is located too high in the stripper 18, the temperature will be too low and the conversion will be incomplete. If the nozzle is located too low in the stripper 18, ethylene will be carried into the catalyst regenerator 30. In some embodiments, the ethanol feed 26 is into the upper half or third of the stripper 18, for example, to obtain high yields. In some embodiments, the ethylene yield at least 50%, at least 70%, at least 85%, or more. Furthermore, it may be advantageous to use two or more nozzles or a distributor to create a uniform distribution to minimize downflow of ethylene and unconverted ethanol into the catalyst regenerator 30.

An FCC effluent 28 including cracked products from the hydrocarbon feed and ethylene from the ethanol feed is withdrawn from the reaction vessel 14. While not shown, the FCC effluent 22 is further processed, for example, separated to form various products, such as an FCC ethylene, C₄₋ product (a light hydrocarbon product), an FCC naphtha, an FCC cycle oil, and/or a bottoms product. The reaction in the reactor 12 also produces a coke that deposits on the FCC catalyst causing it to lose effectiveness. In order to maintain effectiveness of the FCC catalyst, the spent FCC catalyst must be regenerated. As illustrated, the spent FCC catalyst is fed to a catalyst regenerator 30 via spent catalyst line 32, for example, to remove the coke. The catalyst regenerator 30 contains a two-stage cyclones separator 34 that spin the hydrocarbon and catalyst mixture wherein the heavier catalyst slides to the bottom of the reactor and exit the catalyst regenerator through pipe 38 while the hydrocarbon rises from the cyclone to the top of the reactor 30 and exit through 36. As illustrated, the reactor 12 and the catalyst regenerator 30 can be separate vessels. An oxygen-containing gas can be fed to the catalyst regenerator 30. After regeneration, the regenerated FCC catalyst is then fed back to the reactor 12, for example, to the riser 16 via regenerated catalyst line 38.

### Additional Embodiments

Accordingly, the present disclosure provides methods and systems for co-processing a hydrocarbon feed in an FCC system with an ethanol feed. The methods and systems may include any of the various features disclosed herein, including one or more of the following statements.
Embodiment 1. A method of ethanol conversion and cracking hydrocarbons, comprising: introducing a hydrocarbon feed into a riser of a fluid catalytic cracking reactor; introducing ethanol into a stripper of the fluid catalytic cracking reactor; and reacting at least the hydrocarbon feed and the ethanol in the presence of one or more fluid catalytic cracking catalysts in the fluid catalytic cracking reactor to produce at least cracked products and ethylene.
Embodiment 2. The method of Embodiment 1, wherein the ethanol is fed into the upper half of the stripper.
Embodiment 3. The method of Embodiment 1, wherein the ethanol is fed into the upper third of the stripper.
Embodiment 4. The method of any preceding Embodiment, wherein the ethanol is fed into the stripper above a steam feed.
Embodiment 5. The method of any preceding Embodiment, wherein the hydrocarbon feed comprises vacuum gas oil.
Embodiment 6. The method of any preceding Embodiment, wherein the hydrocarbon feed is fed to the FCC at a concentration of 50 wt. % to 95 wt. % based on a total weight of the ethanol and the hydrocarbon feed, and wherein the ethanol is fed to the FCC at a concentration of 5 wt. % to 50 wt. % based on the total weight of the ethanol and the hydrocarbon feed.
Embodiment 7. The method of any preceding Embodiment, wherein the ethanol reacts in the stripper in the presence of the one or more fluid catalytic cracking catalysts, the one or more fluid catalytic cracking catalyst comprises comprising spent catalyst flowing downwardly from a separator section.
Embodiment 8. The method of any preceding Embodiment, wherein the fluid catalytic cracking reactor comprises a riser and reaction vessel, wherein the reaction vessel comprises a separator section and the stripper.
Embodiment 9. The method of any preceding Embodiment, wherein ethanol is fed into the stripper through more than one nozzles.
Embodiment 10. The method of any preceding Embodiment, wherein an ethylene yield is 70% or greater.
Embodiment 11. The method of any preceding Embodiment, wherein an ethylene yield is 85% or greater.
Embodiment 12. The method of any preceding Embodiment, wherein the one or more fluid catalytic cracking catalysts comprise a zeolitic component.
Embodiment 13. The method of any preceding Embodiment, further comprising removing a cracked effluent from the fluid catalytic cracking reactor, wherein the cracked effluent comprises the cracked products and the ethylene; and separating at least a light hydrocarbon product from the cracked effluent, wherein the light hydrocarbon product comprises hydrocarbons having 4 or less carbons.
Embodiment 14. A method of ethanol conversation and cracking hydrocarbons, comprising: introducing a hydrocarbon feed into a riser of a fluid catalytic cracking reactor such that hydrocarbons in the hydrocarbon feed react in the presence of one or more fluid catalytic cracking catalysts to produce at least cracked products; and introducing an ethanol feed into an upper one third of a stripper of the fluid catalytic cracking reactor such that the ethanol reacts in the presence of spent catalyst flowing downwardly from a separator section of the fluid catalytic cracking reactor to produce at least ethylene.
Embodiment 15. The method of Embodiment 14, wherein hydrocarbon feed comprises at least one hydrocarbon feed selected from the group consisting of petroleum crude, an atmospheric residue, a vacuum residue, propane deasphalted residue, a fluid catalytic tower bottom, an atmospheric gas oil, a vacuum gas oil, a coker gas oil, a distillate, a hydrocrackate, a hydrotreated oil, a dewaxed oil, a slack wax, a Fischer-Tropsch wax, a raffinate, and combinations thereof.
Embodiment 16. The method of Embodiment 14 or Embodiment 15, wherein the one or more fluid catalytic cracking catalysts comprises a zeolitic component.
Embodiment 17. The method of claim any one of Embodiments 14 to 16, wherein ethanol is fed into the stripper through more than one nozzles.
Embodiment. 18. A system for ethanol conversion and cracking hydrocarbons, comprising: a first source of a hydrocarbon feed comprising hydrocarbons; a second source of an ethanol feed comprising ethanol; and a fluid catalytic cracking system comprising a fluid catalytic cracking reactor and a catalyst regenerator, wherein the fluid catalytic cracking reactor is fluidically coupled to the catalyst regenerator such that the fluid catalytic cracking reactor receives regenerated catalyst from the catalyst regenerator, wherein the fluid catalytic cracking reactor comprises a riser fluidically coupled to the first source of the hydrocarbon feed; and wherein the fluid catalytic cracking reactor comprises a reactor vessel that comprises a separator section and a stripper, the stripper being fluidically coupled to the second source of the ethanol feed.
Embodiment 19. The system of Embodiment 18, wherein the fluid catalytic cracking reactor comprises a fluid catalytic cracking catalyst comprising a zeolitic component.
Embodiment 20. The system of Embodiment 17 or Embodiment 18, wherein the stripper is further fluidically coupled to a steam feed, the ethanol feed being fluidically coupled in an upper third of the stripper above the steam feed.

To facilitate a better understanding of the present invention, the following examples of certain aspects of some embodiments are given. In no way should the following examples be read to limit, or define, the entire scope of the disclosure.

### Examples

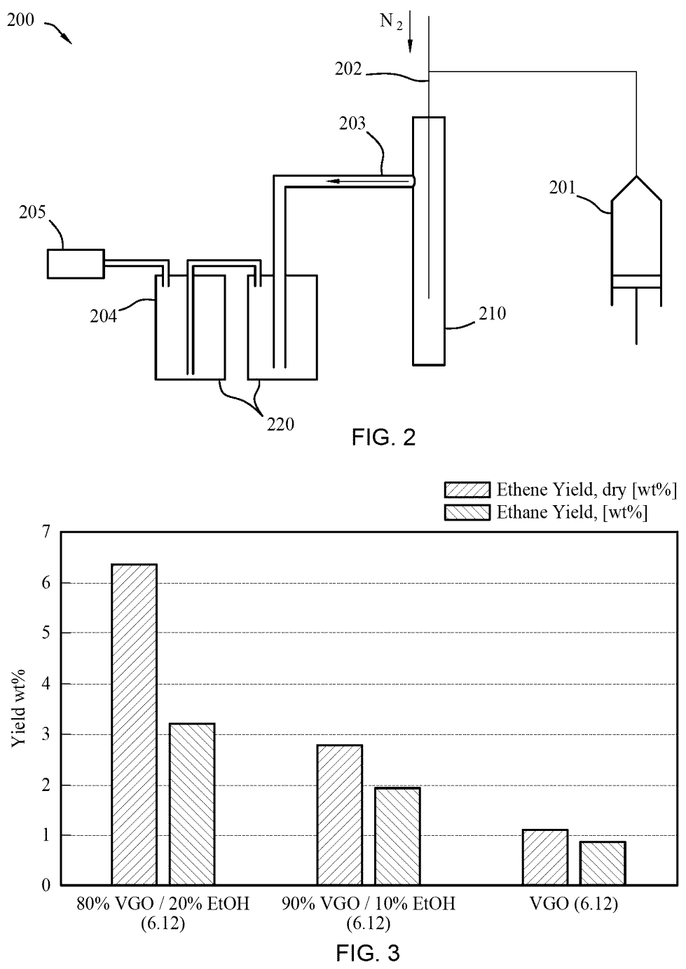

FIG. 2 illustrates a pilot plant unit 200 that was used to test the ethanol to ethylene conversion reaction. The pilot plant unit 200 included a feed 201, a catalyst (not shown), a reactor 210, and a product collection 220. The reactor 210 was an ACE Model R 210 from Kayser Technology, Inc. The catalyst used in the FCC reactor was E-Cat, which is used commercially at refineries.

In operation, the feed 201 was preheated at 80° C. and then injected into the pilot plant metallic reactor 210, with a special motor pump (not shown), through an injector 202. A computer controlled the pumping speed and time. The pilot plant metallic unit 200 was heated by a three-zone furnace (not shown). Following the oil injection, nitrogen was flowed into the reactor 210 in order to drive the products in the exit line 203 of the reactor. At the reactor exit, specially designed receivers (not shown), were connected for the condensation of the liquid products. Gaseous products of the cracking reaction were collected in a cylinder 204 by water displacement. The gaseous products were analyzed with a specially designed micro-gas chromatography (μGC) system 205. Moreover, a Simulated Distillation GC (Agilent 6890), measures the conversion of liquid products. The condenser was also rinsed with dry methanol to remove any condensates product on the walls. The amount of coke, deposited on the catalyst, was measured online with a carbon dioxide (CO₂) analyzer (not shown). The pilot plant unit 200 was fully automated using the control software FIX and thus all operator actions are performed using a PC. The computer system accepted all of the process signals, maintains a digital log of all the variables and also updates the signals.

The following process conditions were used for all tests with the pilot plant 200: (1) reactor 210 temperature of 482° C. to 543° C.; (2) catalyst regenerator (not shown) temperature had a setpoint of 718° C. (process variable of 700° C.); (3) catalyst mass from 4 g to 11 g; (4) a feed rate of 1.82 g/min for 42 s; (5) catalyst stripping time of 340 s; and (6) catalyst to oxygen ratio from 6.12 to 8.63.

A series of tests were conducted to evaluate coprocessing a vacuum gas oil with ethanol in a laboratory-scale FCC reactor 210. The tests were conducted at the above process conditions with a reactor temperature of 543° C., catalyst mass of 7.8 g, and 1.274 g/min feed flow rate. In this example, each test was run 3 times to ensure data reproducibility. The following data are from the first run. Four feeds to the FCC reactor were tested as follows:
1. Feed 1 (Comparative): Vacuum Gas Oil (VGO) only and a catalyst to oil ratio of 6.12.
2. Feed 2 (Comparative): VGO/Ethanol (90/10) and a catalyst to oil ratio of 6.12 and 8.63.
3. Feed 3 (Comparative): VGO/Ethanol (80/20) and a catalyst to oil ratio of 6.12.
4. Feed 4: Ethanol only and a catalyst to oil ratio of 6.12.

The test data for Feeds 1-3 were analyzed to determine conversion and product yield. The results of Feeds 1-3 are shown in FIGS. 3-5.

FIG. 3 is a chart showing the yield of the ethanol conversion to ethene and ethane as a function of the feed. For Feed 1 (no ethanol, just VGO), the ethene yield is around 1 wt % while the ethane yield is just below 1 wt %. However, as ethanol is introduced into the feed in Feed 2 (10% ethanol), the ethene yield rises to almost 3 wt % while the ethane yield is close to 2 wt %. In contrast, when 20% ethanol is used in the feed in Feed 3, ethene yield is over 6 wt %, twice as high as the ethane yield at around 3 wt %.

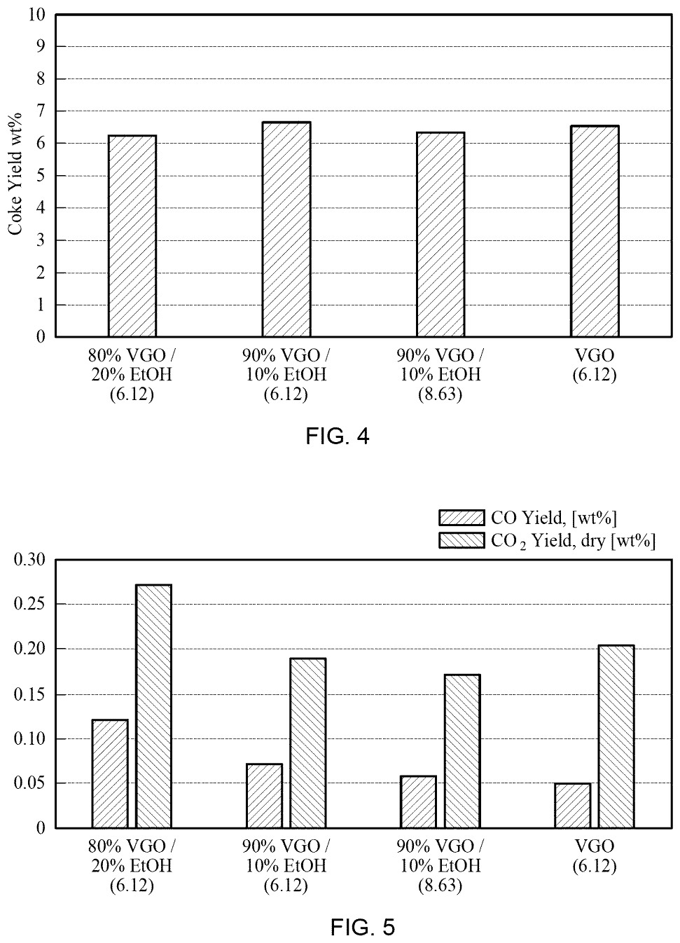

FIG. 4 is a chart of the coke yield as a function of feed and catalyst to oil ratio. The coke yield is in between 6 and 7 wt % for the three feeds and the two catalyst to oil ratios tested.

FIG. 5 is a chart of the carbon monoxide and carbon dioxide yield as a function of feed and of the catalyst to oil ratio. Feed 1 (no ethanol, just VGO) results in a 0.2 wt % carbon dioxide yield and a 0.05 wt % carbon monoxide yield. As ethanol is introduced in Feed 2 (10% ethanol), the yield of carbon dioxide is lowered to 0.17 wt % while the yield of carbon monoxide increases just above 0.05 wt % with a catalyst to oil ratio of 8.63. As the catalyst to oil ratio is lowered to 6.12 with the same amount of ethanol (10%) and VGO (90%), the yield of carbon dioxide raises to close to 0.2 wt % and the yield of carbon monoxide increases to 0.05 wt %. However, increasing the amount of ethanol to 20% leads to an increase to the carbon dioxide yield of 0.27 wt % and a carbon monoxide yield of 0.12 wt %.

Accordingly, the tests for Feeds 1-3 shows that when ethanol was blended with VGO in the pilot plant unit 200, higher yields to ethylene were increased but CO, CO₂, and ethane yields were also increased. The coke yield remains the same regardless of the feed and the catalyst to oil ratio. While the preceding tests for Feeds 1-3 illustrate ethanol conversion in an FCC system, the ethanol is co-fed with VGO. However, example embodiments disclosed herein feed the ethanol into the stripper of the FCC system. In the stripper, spend catalyst flows downward, for example, through a steam stripping section to remove hydrocarbon vapors before the spend catalyst returns to the catalyst regenerator.

To simulate processing of ethanol in a stripper, Feed 4 was tested with three separate catalysts: (1) fresh catalyst (comparative); (2) exitu coked catalyst; and (3) insitu coked catalyst. To prepare the exitu coked catalyst, the fresh catalyst was deactivated by a cyclic metals deactivation unit (CDMU). The exitu coked catalyst had a coke content of 1.67 wt. %. To prepare the insitu coked catalyst, the fresh catalyst was made by exposing the catalyst to Feed 1 (VGO only) at the process conditions provided above. The insitu coked catalyst had a coke content of 1.15 wt. %.

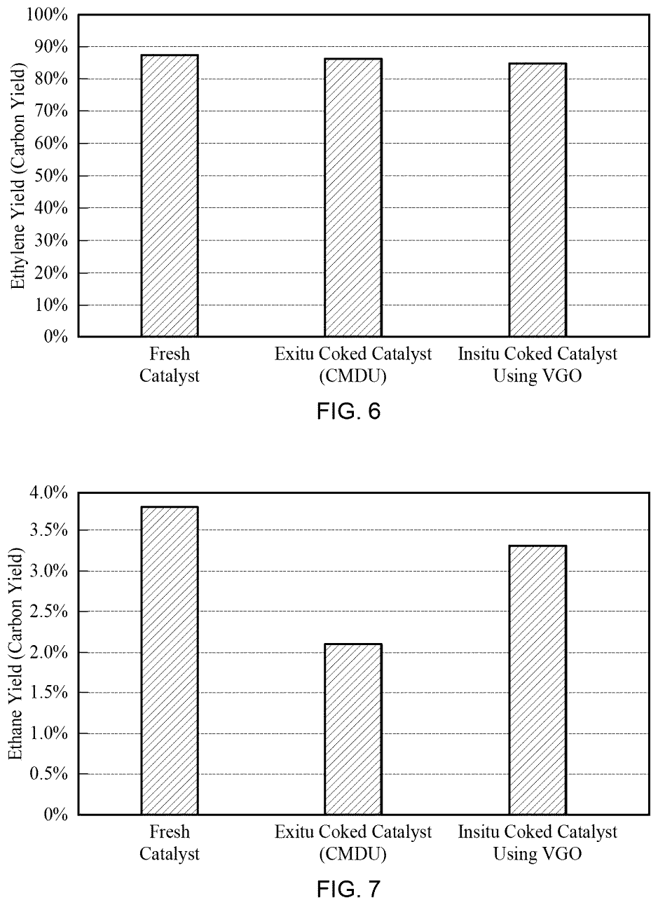

The test data for Feed 4 over the three catalysts were analyzed to determine conversion and product yield. The results of Feeds 1-3 are shown in FIGS. 6-11.

FIG. 6 is a chart of the ethylene yield as a function of catalyst: fresh catalyst (uncoked E-Cat); exitu coked catalyst; and insitu catalyst. The three catalysts show similar ethylene yield (between 85% and 88%). Therefore, the data indicates that spent catalysts are still active in ethanol conversion.

FIG. 7 is a chart of the ethane yield as a function of catalyst: fresh catalyst (uncoked E-Cat); exitu coked catalyst; and insitu catalyst. The three catalysts show low ethane yield (less than 5%). The test with the exitu coked catalyst shows the lowest ethane yield (2%).

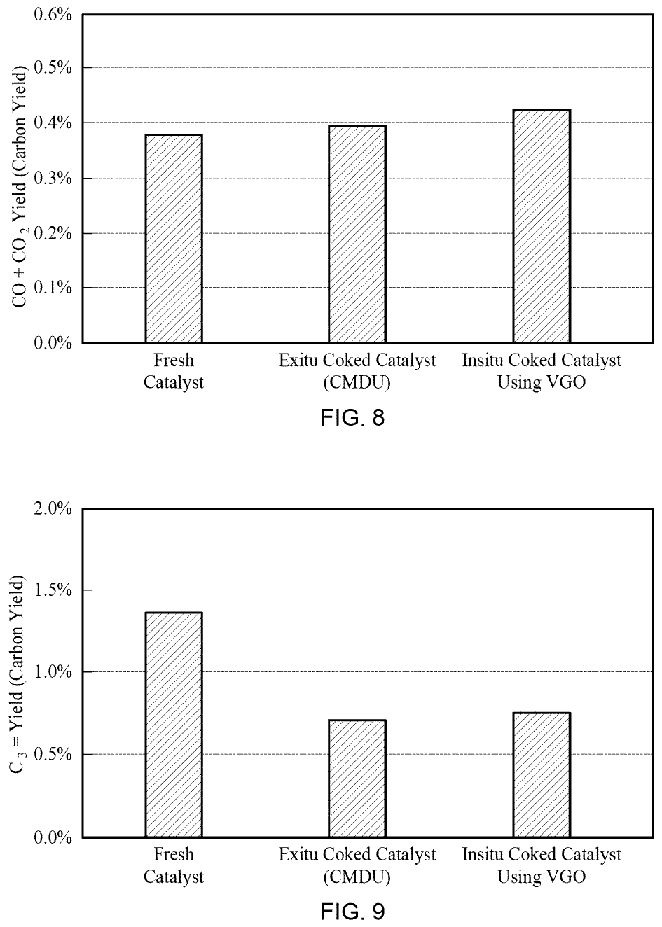

FIG. 8 is a chart of the (CO+CO₂) yield as a function of catalyst: fresh catalyst (uncoked E-Cat); exitu coked catalyst; and insitu catalyst. The three catalysts show low (CO+CO₂) yield (less than 0.5%).

FIG. 9 is a chart of the propylene (C₃) yield as a function of catalyst: fresh catalyst (uncoked E-Cat); exitu coked catalyst; and insitu catalyst. The three catalysts show a yield of less than 1.5% propylene (C₃) with the fresh catalyst helping to produce the highest quantity of propylene and the exitu coked catalyst producing the lowest propylene yield.

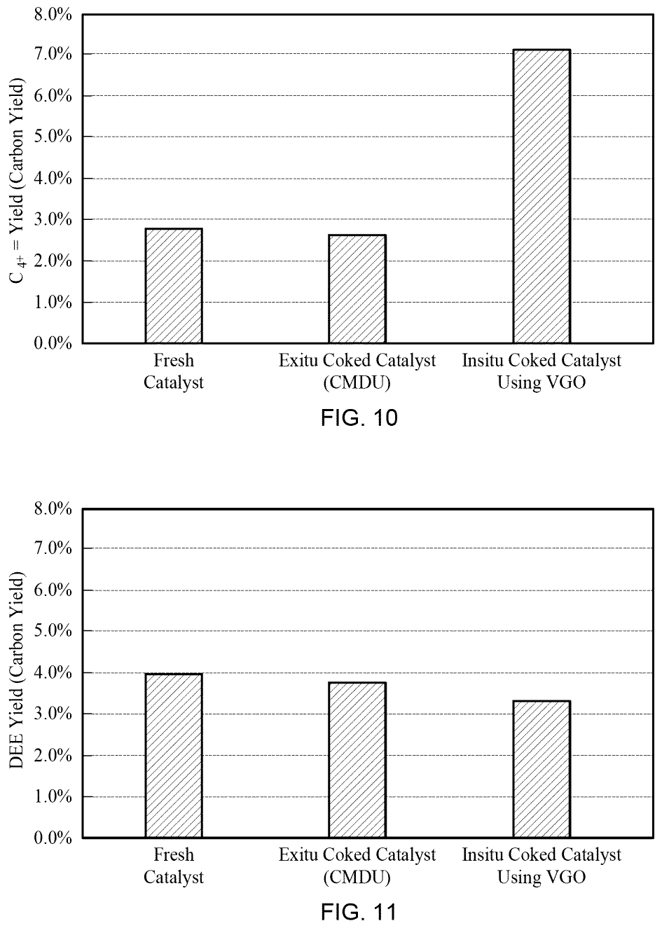

FIG. 10 is a chart of the other hydrocarbon produced than ethylene (C₄₊) yield as a function of catalyst: fresh catalyst (uncoked E-Cat); exitu coked catalyst; and insitu catalyst. The yield is around 7% with the insitu coked catalyst due to residue from the coking catalyst while the fresh catalyst and the exitu coked catalyst yield less than 3% C₄₊.

FIG. 11 is a chart of liquid analysis indicating that less than 4% of diethyl ether was formed in addition to water with the fresh catalyst giving 4% while the insitu coked catalyst giving closer to 3%.

Accordingly, the test with Feed 4 over the three different catalysts shows that ethanol can be converted to ethylene at the stripper conditions in high yield to ethylene (88%) with only 4% of the feed was converted to ethane. In addition, the coke yield was very low (less than 2%) with corresponding and low yield to CO—CO₂, indicating no decarbonylation. The thermodynamics calculations indicated that the major by-product, diethyl ether (DEE), is completely converted to ethylene at temperatures above 300° C.

While the disclosure has been described with respect to a number of embodiments and examples, those skilled in the art, having benefit of this disclosure, will appreciate that other embodiments can be devised which do not depart from the scope and spirit of the disclosure as disclosed herein. Although individual embodiments are discussed, the present disclosure covers all combinations of all those embodiments.

While compositions, methods, and processes are described herein in terms of “comprising,” “containing,” “having,” or “including” various components or steps, the compositions and methods can also “consist essentially of” or “consist of” the various components and steps. The phrases, unless otherwise specified, “consists essentially of” and “consisting essentially of” do not exclude the presence of other steps, elements, or materials, whether or not, specifically mentioned in this specification, so long as such steps, elements, or materials, do not affect the basic and novel characteristics of the disclosure, additionally, they do not exclude impurities and variances normally associated with the elements and materials used.

All numerical values within the detailed description are modified by “about” the indicated value, and take into account experimental error and variations that would be expected by a person having ordinary skill in the art.

Many alterations, modifications, and variations will be apparent to those skilled in the art in light of the foregoing description without departing from the spirit or scope of the present disclosure and that when numerical lower limits and numerical upper limits are listed herein, ranges from any lower limit to any upper limit are contemplated.

What is claimed is:

1. A method of ethanol conversion and cracking hydrocarbons, comprising:
introducing a hydrocarbon feed into a riser of a fluid catalytic cracking reactor;
introducing ethanol into a stripper of the fluid catalytic cracking reactor; and
reacting at least the hydrocarbon feed and the ethanol in the presence of one or more fluid catalytic cracking catalysts in the fluid catalytic cracking reactor to produce at least cracked products and ethylene.

2. The method of claim 1, wherein the ethanol is fed into the upper half of the stripper.

3. The method of claim 1, wherein the ethanol is fed into the upper third of the stripper.

4. The method of claim 1, wherein the ethanol is fed into the stripper above a steam feed.

5. The method of claim 1, wherein the hydrocarbon feed comprises vacuum gas oil.

6. The method of claim 1, wherein the hydrocarbon feed is fed to the FCC at a concentration of about 50 wt. % to about 95 wt. % based on a total weight of the ethanol and the hydrocarbon feed, and wherein the ethanol is fed to the FCC at a concentration of about 5 wt. % to about 50 wt. % based on the total weight of the ethanol and the hydrocarbon feed.

7. The method of claim 1, wherein the ethanol reacts in the stripper in the presence of the one or more fluid catalytic cracking catalysts, the one or more fluid catalytic cracking catalyst comprises comprising spent catalyst flowing downwardly from a separator section.

8. The method of claim 1, wherein the fluid catalytic cracking reactor comprises a riser and reaction vessel, wherein the reaction vessel comprises a separator section and the stripper.

9. The method of claim 1, wherein ethanol is fed into the stripper through more than one nozzles.

10. The method of claim 1, wherein an ethylene yield is about 70% or greater.

11. The method of claim 1, wherein an ethylene yield is about 85% or greater.

12. The method of claim 1, wherein the one or more fluid catalytic cracking catalysts comprise a zeolitic component.

13. The method of claim 1, further comprising removing a cracked effluent from the fluid catalytic cracking reactor, wherein the cracked effluent comprises the cracked products and the ethylene; and separating at least a light hydrocarbon product from the cracked effluent, wherein the light hydrocarbon product comprises hydrocarbons having 4 or less carbons.

14. A method of ethanol conversation and cracking hydrocarbons, comprising:
introducing a hydrocarbon feed into a riser of a fluid catalytic cracking reactor such that hydrocarbons in the hydrocarbon feed react in the presence of one or more fluid catalytic cracking catalysts to produce at least cracked products; and
introducing an ethanol feed into an upper one third of a stripper of the fluid catalytic cracking reactor such that the ethanol reacts in the presence of spent catalyst flowing downwardly from a separator section of the fluid catalytic cracking reactor to produce at least ethylene.

15. The method of claim 14, wherein hydrocarbon feed comprises at least one hydrocarbon feed selected from the group consisting of petroleum crude, an atmospheric residue, a vacuum residue, propane deasphalted residue, a fluid catalytic tower bottom, an atmospheric gas oil, a vacuum gas oil, a coker gas oil, a distillate, a hydrocrackate, a hydrotreated oil, a dewaxed oil, a slack wax, a Fischer-Tropsch wax, a raffinate, and combinations thereof.

16. The method of claim 14, wherein the one or more fluid catalytic cracking catalysts comprises a zeolitic component.

17. The method of claim 13, wherein ethanol is fed into the stripper through more than one nozzles.

18. A system for ethanol conversion and cracking hydrocarbons, comprising:
a first source of a hydrocarbon feed comprising hydrocarbons;
a second source of an ethanol feed comprising ethanol; and
a fluid catalytic cracking system comprising a fluid catalytic cracking reactor and a catalyst regenerator,
wherein the fluid catalytic cracking reactor is fluidically coupled to the catalyst regenerator such that the fluid catalytic cracking reactor receives regenerated catalyst from the catalyst regenerator,
wherein the fluid catalytic cracking reactor comprises a riser fluidically coupled to the first source of the hydrocarbon feed; and
wherein the fluid catalytic cracking reactor comprises a reactor vessel that comprises a separator section and a stripper, the stripper being fluidically coupled to the second source of the ethanol feed.

19. The system of claim 18, wherein the fluid catalytic cracking reactor comprises a fluid catalytic cracking catalyst comprising a zeolitic component.

20. The system of claim 18, wherein the stripper is further fluidically coupled to a steam feed, the ethanol feed being fluidically coupled in an upper third of the stripper above the steam feed.
